(19) United States
(12) Patent Application Publication
(10) Pub. No.: US 2022/0104687 A1
(43) Pub. Date: Apr. 7, 2022

(54) USE OF COMPUTER VISION TO DETERMINE ANATOMICAL STRUCTURE PATHS

(71) Applicant: Asensus Surgical US, Inc., Durham, NC (US)

(72) Inventors: Kevin Andrew Hufford, Durham, NC (US); Tal Nir, Durham, NC (US); Lior Alpert, Durham, NC (US)

(21) Appl. No.: 17/495,803

(22) Filed: Oct. 6, 2021

Related U.S. Application Data
(60) Provisional application No. 63/088,404, filed on Oct. 6, 2020.

(51) Int. Cl. A61B 1/00; A61B 1/04; A61B 34/20; A61B 34/32

(52) U.S. Cl. CPC A61B 1/00009; A61B 1/043; A61B 2034/301; A61B 34/20; A61B 34/32; A61B 1/00045

(57) ABSTRACT

Surgical assistance is provided in the form of an image of the surgical site that has overlays marking anatomical structures of interest to the surgeon. The system uses computer vision to detect the anatomical structures of interest that are visible to the camera, and predicts the location, shape and/or orientation of portions of the anatomical structures that are not visible to the camera. The overlays mark both the visible portions of the anatomical structures and the predicted location, shape and orientation of the invisible portions.

### BACKGROUND

This application relates to the use of computer vision to recognize anatomical features within a surgical site. In many procedures, it is necessary to track anatomical structures present within the surgical site. Some of those anatomical structures are ones that follow a path within the body. Examples include ureters, ducts, blood vessels, nerves, etc.

Sometimes the complete entire path of the structure may not be visible in the endoscopic view at once. One or more portions of the path may be occluded by organs or other tissue layers. During the course of some procedures, occluded portion(s) of the path may be exposed gradually by surgical dissection.

The concepts disclosed in this application aid the surgeon by helping to identify and track the path of an anatomical structure. This enhances the surgeon's awareness of structures that may only be differentiable via context clues such as their source or destination, and helps the surgeon undertake measures to avoid damaging fragile structures.

### BRIEF DESCRIPTION OF THE DRAWINGS

FIG. 1 is a block diagram showing an example of a system according to the disclosed concepts;

FIG. 2 shows an endoscopic image display displaying a cystic duct and an overlay marking the cystic duct;

FIGS. 3-6 are a sequence of drawings graphically depicting a method in which parts of an anatomic structure are detected by a system and marked with overlays, and in which the pathway of the invisible parts is predicted and displayed.

### DETAILED DESCRIPTION

System

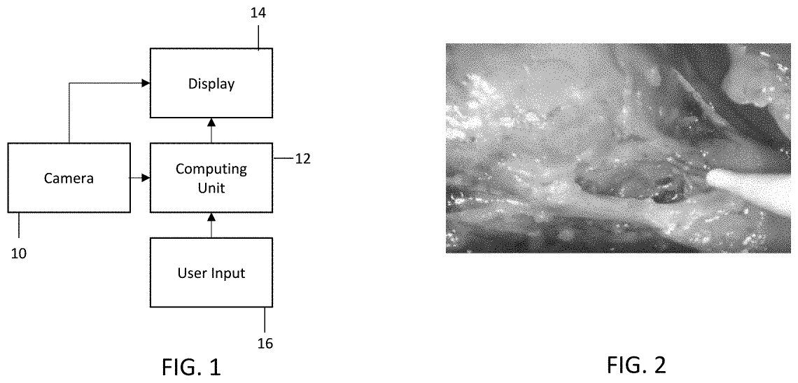

A system useful for performing the disclosed methods, as depicted in FIG. 1, may comprise a camera 10, a computing unit 12, a display 14, and, preferably, one or more user input devices 16. The system is intended to be used during surgical procedures in which instruments are manipulated at a surgical site for treatment or diagnostic purposes. The instruments may be the type that are manually moved by a surgeon. They might also be part of a robot-assisted surgical system in which instruments are maneuvered by robotic components, either in response to input given to the surgical system by a surgeon, semi-autonomously (with a user providing supervisory oversight) or autonomously.

In still other implementations, this recognition and tracking is a component of a fully autonomous surgical procedure.

The camera 10 is one suitable for capturing images of the surgical site within a body cavity. It may be a 3D or 2D endoscopic or laparoscopic camera. Where it is desirable to use image data to detect movement or positioning of instruments or tissue in three dimensions, configurations allowing 3D data to be captured or derived are used (e.g., a stereo/3D camera, or a 2D camera with software and/or hardware configured to permit depth information to be determined or derived).

The computing unit 12 is configured to receive the images/video from the camera and input from the user input device(s). If the system is to be used in conjunction with a robot-assisted surgical system in which surgical instruments are maneuvered within the surgical space using one or more robotic components (e.g. robotic manipulators that move the instruments and/or camera, and/or robotic actuators that articulate joints, or cause bending, of the instrument or camera shaft) the system may optionally be configured so that the computing unit also receives kinematic information from such robotic components 18 for use in recognizing procedural steps or events as described in this application.

An algorithm stored in memory accessible by the computing unit is executable to, depending on the particular application, use the image data to perform one or more of the functions described with respect to the below-described embodiments.

The system may include one or more user input devices 16. When included, a variety of different types of user input devices may be used alone or in combination. Examples include, but are not limited to, eye tracking devices, head tracking devices, touch screen displays, mouse-type devices, voice input devices, foot pedals, or switches. Various movements of an input handle used to direct movement of a component of a surgical robotic system may be received as input (e.g., handle manipulation, joystick, finger wheel or knob, touch surface, button press). Another form of input may include manual or robotic manipulation of a surgical instrument having a tip or other part that is tracked using image processing methods when the system is in an input-delivering mode, so that it may function as a mouse, pointer and/or stylus when moved in the imaging field, etc. Input devices of the types listed are often used in combination with a second, confirmatory, form of input device allowing the user to enter or confirm (e.g., a switch, voice input device, button, icon to press on a touch screen, etc., as non-limiting examples).

The system is configured to perform one or more of the following functions:
Using computer vision to recognize path-like structures and tag them
Marking recognized structures with overlays
Extending the overlays as additional regions of the structures are recognized, which may occur as a result of exposure of the additional regions from surgical dissection or other techniques
Entering of tagged structures into a repository/database
Tracking of tagged structures through camera movements in which they may go offscreen
Use of predictive algorithms to determine connectedness between path-like structures
Use of context clues to determine the identity of anatomical structures—not only their type, but also their use

### EXAMPLES

A first example is given in the context of a cholecystectomy, a procedure during which it is necessary for the surgeon to be aware of the cystic duct and the common bile duct. During cholecystectomy, the cystic duct is clipped and cut, but the common bile duct cannot be cut. During the course of the procedure, the cystic duct is gradually exposed via dissection. The system uses computer vision to recognize the cystic duct, and an overlay is generated as shown in FIG. 2 to mark the cystic duct for the user. As the user continues to expose more of the cystic duct, the overlay is extended to additionally mark the newly exposed sections.

A second example relates to a hysterectomy or colorectal procedure. During these procedures, the surgeon wants to maintain an awareness of the location of the ureter to avoid inadvertent injure to it. However, the entire path of the ureter may not be visible at all times. In this case, the system displays overlays marking the portions of the ureter recognized by the system using computer vision, as shown in FIG. 3. More particularly, computer vision is applied to images captured of the surgical site, and the ureter is identified and tagged. Techniques by which computer vision can be used to identify structures at an operative site are described in commonly owned U.S. application Ser. No. 17/035,534, “Method and System for Providing Real Time Surgical Site Measurements,” and US2020/0205991, “Instrument Path Guidance Using Visualization and Fluorescence”, each of which is incorporated herein by reference. The system may automatically seek the structures, or the user may give input identifying parts of the structures to the system, or the user may give input instructing the system to identify structures within a defined region. Features of these types are described in U.S. application Ser. No. 17/035,534, and in U.S. 63/048,180, entitled Automatic Tracking of Target Treatment Sites Within Patient Anatomy, both of which are incorporated herein by reference. Although this method is described with respect to the ureter, it may also be used to identify and tag other path-like structures such as blood vessels etc.

Pre-operative imaging may be optionally used to identify and the tag structures, with live correlation then used during surgery to correlate those structures with the real time endoscopic view.

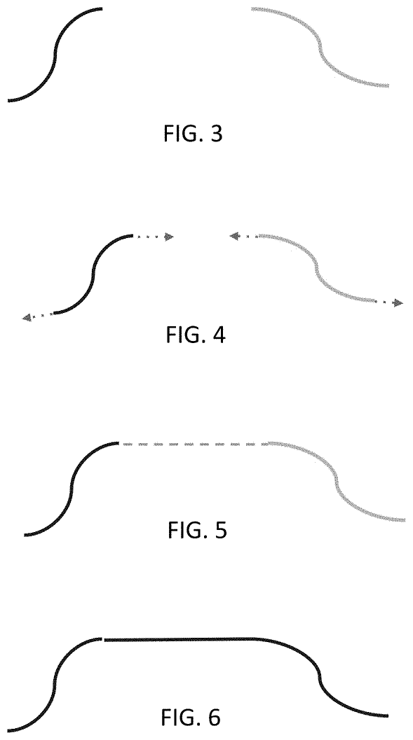

With regard to the portions of the ureter or other path-like structure that cannot be detected by the system, the system predicts that path of the structure based on the detected portions, and, optionally, other information known or learned by the system. The system displays its predictive path as an overlay on the endoscopic display so as to can help to avoid inadvertent injury to it. This is illustrated in FIG. 4, in which the nominal directions of the visible portions of the structures are identified and used to search for potential connections between those portions.

Referring to FIG. 5, potential connection between the portions of the structures are identified. The potential connections may be displayed to the user as overlays on the image display. Alternatively, the user may draw the connection or otherwise inform the system of the connection. (Using any of the input devices described above, or a heads up display, eye tracking, input device, floating handles, gestures, haptic input device, touchscreen, tablet, stylus, etc.)

With increased confidence or with user direction, the path connecting what is now believed or known to be the same structure(s) or at least connected structures may be confirmed and tracked. See FIG. 6. These may be presented to the user as a controllable overlay on the endoscopic image display.

Although the paths shown above are straight lines, the predicted shape may have any shape, including straight-line, splines, arcs, etc. or any combination thereof.

The system may make use of active contour models/snake models and their properties to define an acceptable path/potential connectivity criteria. Other anatomical landmarks recognized by the system or identified to the system by the user may be taken into account by the system in predicting pathways. Definition of pathways may also be performed with reference to other instruments. See, for example, commonly owned U.S. Ser. No. 16/733,147 “Guidance of Robotically Controlled Instruments Along Paths Defined with Reference to Auxiliary Instruments”, incorporated by reference.

With the paths predicted or identified, the following additional functions may be optionally be performed:
The predicted/identified paths are marked with overlays to allow the user to easily differentiate between similar-looking structures/tissue
The system may define “no-fly” zones relative to the predicted/identified paths. The boundaries of the zones may be displayed as overlays to alert the user to stay within or outside the zones. Additionally, or alternatively, the system may prevent robotically manipulated surgical instruments from being moved within the defined zones or structures or allow robotically manipulated surgical instruments to only work within defined zones. See, for example, co-pending U.S. Ser. No. 16/237,444 “System and Method for Controlling a Robotic Surgical System Based on Identified Structures” which is incorporated herein by reference.
Overlays and/or prompts may be displayed alerting the user as to which of multiple similarly-appearing structures are to be acted on (e.g. in the cystic duct/common bile duct example, “clip this” or “don't clip this”)

Machine learning algorithms may be employed to help the system to provide increasingly accurate recommendations over time, as the accuracy of predictions are confirmed to the system and used to train the algorithms.

All patents and applications described herein, including for purposes of priority, are incorporated by reference.

1. A system comprising:
a camera positionable to capture image data corresponding to a treatment site that includes an anatomical structure having a pathway, the anatomical structure having a first portion visible at the treatment site and a second portion obscured at the treatment site;
at least one processor and at least one memory, the at least one memory storing instructions executable by said at least one processor to:
identify at least one portion of the anatomical structure within images captured using the camera; and
predict a pathway followed by the second portion at the treatment site; and
provide output to a user identifying the predicted pathway of the second portion.

2. The system of claim 1, wherein the first portion is a portion visible under fluorescence.

3. The system of claim 2, wherein the output includes a display of an overlay indicating the predicted pathway.
